(19) United States
(12) Patent Application Publication
(10) Pub. No.: US 2012/0259182 A1
(43) Pub. Date: Oct. 11, 2012

(54) METHOD AND APPARATUS FOR DETECTING PEAK FROM BIOLOGICAL SIGNAL

(76) Inventors: Youn-ho KIM, Hwaseong-si (KR); Jae-min KANG, Seoul (KR); Kun-soo SHIN, Seongnam-si (KR); Tak-hyung LEE, Seoul (KR)

(21) Appl. No.: 13/437,104

(22) Filed: Apr. 2, 2012

(30) Foreign Application Priority Data
Apr. 5, 2011 (KR) 10-2011-0031282

(51) Int. Cl. A61B 5/05; A61B 5/11

(52) U.S. Cl. 600/301

(57) ABSTRACT

A peak detecting method is provided. The peak detecting method includes receiving a biological signal that represents an electrical characteristic difference between electrodes attached to a patient, receiving an activity signal that represents an amount of activity of the patient from an activity sensor detecting activity of the patient, detecting at least one of a plurality of peaks in the input biological signal on the basis of the activity signal, and outputting information regarding the detected peak.

### CROSS-REFERENCE TO RELATED APPLICATIONS

This application claims the benefit under 35 U.S.C. §119(a) of Korean Patent Application No. 10-2011-0031282, filed on Apr. 5, 2011, in the Korean Intellectual Property Office, the entire disclosure of which is incorporated herein by reference for all purposes.

### BACKGROUND

1. Field

The following description relates to a method and apparatus for detecting a peak from a biological signal.

2. Description of the Related Art

As interest in ubiquitous-health has increased, techniques for monitoring and analyzing vital signs of a patient in daily life are more desired. Representative applied techniques for monitoring and analyzing vital signals in daily life include an ElectroCardioGraphy (ECG) measurement module using a fiber-type electrode and a wrist-type, glove-type, or ring-type heart rate detection module. One of the benefits of these applied techniques is placed on usage and portability in combination with miniaturization and a wire/wireless communication mode. Meanwhile, other aspects of techniques for accurately detecting a peak of a biological signal include a capability for monitoring and analyzing vital signs of a patient.

### SUMMARY

According to an aspect, a peak detecting method is provided. The peak detecting method includes receiving a biological signal that represents an electrical characteristic difference between electrodes attached to a patient, receiving an activity signal that represents an amount of activity of the patient from an activity sensor detecting activity of the patient, detecting at least one of a plurality of peaks in the input biological signal on the basis of the activity signal, and outputting information regarding the detected peak.

The detecting of the at least one of the plurality of peaks may include determining a time interval between one of previously-detected peaks and each of the plurality of peaks, selecting one of time intervals of the plurality of peaks on the basis of the activity signal, and detecting a peak of the selected time interval from among the plurality of peaks.

The detecting of the at least one of the plurality of peaks may include determining a pattern that represents a change in an amount of activity of the patient on the basis of a plurality of activity signals including the activity signal, and selecting one of the time intervals of the plurality of peaks on the basis of the determined pattern.

The detecting of the at least one of the plurality of peaks may include in response to it being determined on the basis of the plurality of activity signals including the activity signal that the pattern represents an increased amount of activity of the patient, determining a previous time interval by using time intervals between previously-detected peaks, and selecting one time interval that is less than the previous time interval from among the time intervals of the plurality of peaks.

The detecting of the at least one of the plurality of peaks may include in response to it being determined on the basis of the plurality of activity signals including the activity signal that the pattern represents a decreased amount of activity of the patient, determining a previous time interval by using the time intervals between previously-detected peaks, and selecting one time interval that is greater than the previous time interval from among the time intervals of the plurality of peaks.

The previous time interval may be an average of the time intervals between the previously-detected peaks.

The detecting of the at least one of the plurality of peaks may include determining a pattern that represent a time interval change by using time intervals of previously-detected peaks, selecting one of the time intervals of the plurality of peaks on the basis of the pattern that represents the change in the amount of activity of the patient and the pattern that represents the time interval change, and detecting a peak of the time interval selected from the plurality of peaks.

The detecting of the at least one of the plurality of peaks may include determining a threshold by using at least one of amplitudes of previously-detected peaks, determining a variable to be applied to the threshold on the basis of the activity signal, and detecting at least one of the plurality of peaks on the basis of the threshold and the variable.

The detecting of the at least one of the plurality of peaks may include in response to it is determined on the basis of the activity signal that an amount of activity of the patient is equal to or greater than a predetermined amount, determining the variable to reduce the threshold.

The detecting of the at least one of the plurality of peaks may include determining an amount of activity of the patient on the basis of the activity signal, in response to the amount of activity of the patient being equal to or greater than a predetermined amount, determining a threshold by using at least one of amplitudes of previously-detected peaks, determining a variable to be applied to the threshold on the basis of the activity signal, and detecting at least one of the plurality of peaks on the basis of the threshold and the variable, and in response to the amount of activity of the patient being less than the predetermined amount, determining a threshold by using at least one of amplitudes of previously-detected peaks and detecting at least one of the plurality of peaks on the basis of the threshold.

The detecting of the at least one of the plurality of peaks may include in response to it being determined on the basis of the activity signal that the amount of activity of the patient is equal to or greater than a predetermined amount, determining a threshold by using at least one of amplitudes of previously-detected peaks, determining a sub-threshold less than the threshold by using the threshold, and detecting at least one of the plurality of peaks on the basis of the threshold and the sub-threshold.

The detecting of the at least one of the plurality of peaks may include in response to it being determined on the basis of the activity signal that the amount of activity of the patient is equal to or greater than a predetermined amount, determining a threshold by using at least one of amplitudes of previously-detected peaks, and determining a sub-threshold less than the threshold by using the threshold, in response to one of the plurality of peaks having an amplitude equal to or greater than the sub-threshold, storing the one peak as a candidate detection peak, in response to the one peak having an amplitude equal to or greater than the threshold, detecting the one peak, and in response to the one peak having an amplitude less than the threshold, detecting one of a plurality of candidate detection peaks including the candidate detection peak.

The storing of the one peak as the candidate detection peak may include in response to one peak having an amplitude equal to or greater than the sub-threshold occurs, storing the one peak as the candidate detection peak in real time.

The detecting of the one of the plurality of candidate detection peaks may include determining a pattern that represents a change in the amount of activity of the patient on the basis of a plurality of activity signals including the activity signal, and determining one of the plurality of candidate detection peaks on the basis of the determined pattern.

The detecting of the one of the plurality of candidate detection peaks may include determining a pattern that represents a time interval change by using time intervals between previously-detected peaks, and detecting one of the plurality of candidate detection peaks on the basis of the pattern that represents the change in the amount of activity of the patient and the pattern that represents the time interval change.

The detecting of the one of the plurality of candidate detection peaks may include in response to the one peak being less than the threshold, detecting one of a plurality of candidate detection peaks including the candidate detection peak in consideration of a time interval between the one peak and a previously-detected peak.

The detecting of the one of the plurality of peaks may include determining an amount of activity of the patient on the basis of the activity signal, in response to the amount of activity of the patient being equal to or greater than a predetermined amount, determining a threshold by using at least one of amplitudes of previously-detected peaks, determining a sub-threshold less than the threshold by using the threshold, and detecting at least one of the plurality of peaks on the basis of the threshold and the sub-threshold, and in response to the amount of activity of the patient being less than the predetermined amount, determining a threshold by using at least one of amplitudes of previously-detected peaks and detecting at least one of the plurality of peaks on the basis of the threshold.

The detecting of the one of the plurality of peaks comprises selecting at least one effective peak from among a plurality of peaks in the input biological signal on the basis of the activity signal.

A non-transitory computer readable recording medium on which a program for executing the method may be stored.

The activity signal may be separate from the input biological signal.

The activity signal may represent acceleration, vibration, or impact.

According to another aspect, a peak detecting device is provided. The peak detecting device includes a biological signal input unit configured to receive a biological signal that represents an electrical characteristic difference between electrodes attached to a patient, an activity signal input unit configured to receive an activity signal that represents an amount of activity of the patient from an activity sensor detecting activity of the patient, a peak detecting unit configured to detect at least one of a plurality of peaks in the input biological signal on the basis of the activity signal, and an output unit configured to output information regarding the detected peak.

According to another aspect, a medical device is provided. The medical device includes a peak detecting unit including an activity signal input unit configured to receive an activity signal that represents an amount of activity of a patient, and a peak detector configured to detect at least one of a plurality of peaks in an input biological signal on the basis of the activity signal.

The medical device may further include a display configured to display the information regarding the at least one detected peak.

Other features and aspects may be apparent from the following detailed description, the drawings, and the claims.

### BRIEF DESCRIPTION OF THE DRAWINGS

FIG. 1 is a diagram illustrating an example of a biological signal measuring system;

FIG. 2 is a diagram illustrating an example of a peak detecting device shown in FIG. 1;

FIG. 3 is a flowchart illustrating an example of operations in response to a peak detecting unit of FIG. 2 detecting a peak in consideration of an activity signal;

FIG. 4 is a view illustrating an example of a biological signal;

FIG. 5 is a view illustrating examples of an activity signal and an activity amount;

FIG. 6 is a flowchart illustrating an example of operations in response to the peak detecting unit of FIG. 2 detecting a peak in consideration of an activity signal;

FIG. 7 is a view illustrating examples of a pattern representing changes in the amount of activity of a patient being examined, a pattern representing time intervals, and detected peaks;

FIG. 8 is a flowchart illustrating another example of operations in response to the peak detecting unit detecting a peak in consideration of an activity signal;

FIG. 9 is a flowchart illustrating another example of operations in response to the peak detecting unit of FIG. 2 detecting a peak in consideration of an activity signal;

FIG. 10 is a view illustrating another example of a biological signal;

FIG. 11 is a flowchart illustrating another example of operations in response to the peak detecting unit detecting a peak in consideration of an activity signal;

FIG. 12 is a view illustrating another example of a biological signal;

FIG. 13 is a flowchart illustrating another example of operations in response to the peak detecting unit detecting a peak in consideration of an activity signal; and

FIG. 14 is a flowchart illustrating an example of a peak detecting method.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

Hereinafter, examples will be described with reference to the accompanying drawings.

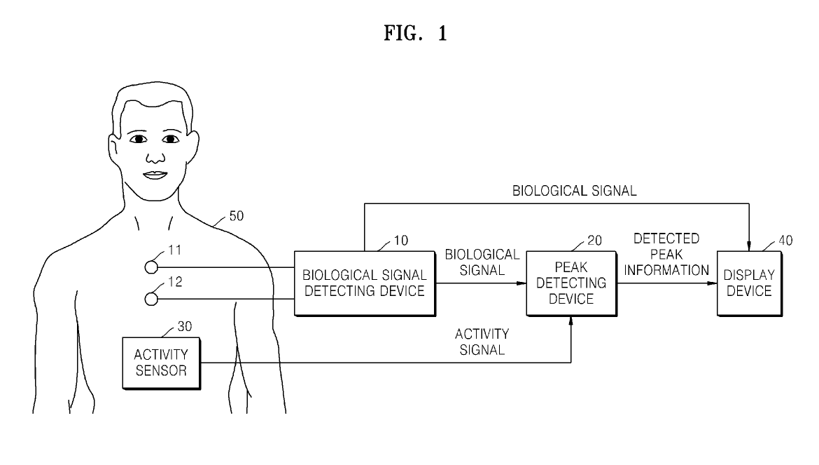

FIG. 1 is a diagram illustrating an example of a biological signal measuring system. Referring to FIG. 1, the biological signal measuring system of FIG. 1 includes a biological signal detecting device 10, a peak detecting device 20, an activity sensor 30, and a display device 40. The biological signal detecting device 10 detects a biological signal by detecting an electrical characteristic difference between electrodes 11 and 12 attached to skin of a patient 50. For example, the electrodes 11 and 12 may be attached to the skin of the patient 50. The electrical characteristic difference between the electrodes 11 and 12 corresponds with an electrical interface between the electrodes 11 and 12 and the skin of the patient 50. The biological signal detecting device 10 detects the electrical characteristic difference. As an example, the electrical characteristic may be an electric potential, and the electrical characteristic difference may be an electric potential difference.

The biological signal measuring device 10 generates a biological signal based on the electrical characteristic difference between the electrodes 11 and 12. The biological signal measuring device 10 may generate a biological signal by signal-processing the detected electrical characteristic difference. The signal-processing of the biological signal may include noise-filtering the detected signals, amplifying the detected signals, and converting the amplified analog signals into digital signals. Accordingly, the biological signal measuring device 10 may further include an amplifier, an analog to digital (A/D) converter, an operator, and a noise filter.

The biological signal detected by the biological signal detecting device 10 is delivered to the display device 40 to display the biological signal. Examples of the display device 40 include a screen or paper. Additionally, the biological signal detected by the biological signal detecting device 10 may also be delivered to the peak detecting device 20. The peak detecting device 20 detects a peak of the biological signal input from the biological signal detecting device 10 and delivers information regarding the detected peak to the display device 40. The display device 40 may display the detected peak based on the biological signal.

Detecting an effective peak from the biological signal may be useful for analyzing the biological signal. For example, in response to the biological signal relating to an ElectroCardioGraphy (ECG) signal, a detected peak from the ECG signal may help analyze a heart rate of the patient 50. An effective peak detected from an ECG signal may correspond to an R peak.

Meanwhile, activity of the patient 50 may contribute to a detection error analyzing an effective peak from a biological signal. For example, an introduction of noise due to activity of the patient 50 may cause peak detection errors of a biological signal and may lead to an incorrect heart rate measurement. As an example, in response to the peak being detected by a portable device, the possibility of detection errors due to activity of the patient 50 may be increased. The peak detecting device 20 may detect a peak of a biological signal by taking into consideration the activity of the patient 50. Examples to be described illustrate a method of detecting a peak of a biological signal in consideration of the activity of the patient 50.

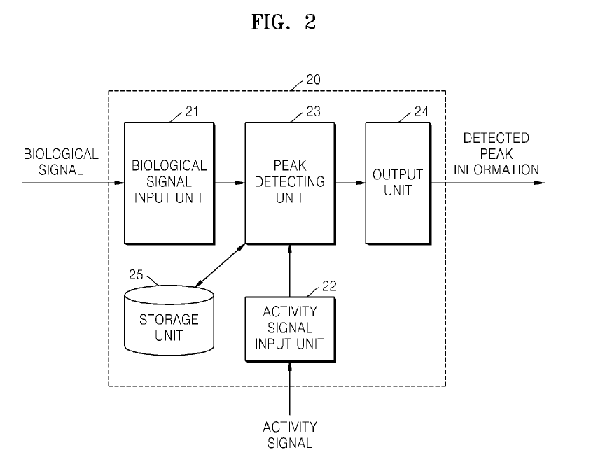

FIG. 2 is a diagram 20 illustrating an example of the peak detecting device 20 shown in FIG. 1. Referring to FIG. 2, the peak detecting device 20 includes a biological signal input unit 21, an activity signal input unit 22, a peak detecting unit 23, an output unit 24, and a storage unit 25. The biological signal input unit 21 receives a biological signal from the biological signal detecting device 10 and delivers the received biological signal to the peak detecting unit 23. The biological signal may represent an electrical characteristic difference between the electrodes 11 and 12 attached to the skin of the patient 50. Additionally, as one example, the biological signal may correspond to an ECG signal. As another aspect, in addition to the ECG signal, the biological signal may further include a brain wave signal and an electromyogram signal. The brain wave signal and the electromyogram signal may also be electrically detected from the skin of the patient 50.

The activity signal input unit 22 receives an activity signal from the activity sensor 30 and delivers the received activity signal to the peak detecting unit 23. This activity signal may correspond to an amount of activity of the patient 50 represented by a numerical value. For example, the amount of activity of the patient 50 may be numerically expressed as a value between 0 and 100. Additionally, this activity signal may vary between 0 and 100 over time.

The activity sensor 30 detects activity of the patient 50, generates an activity signal corresponding to the detected amount of activity and delivers the generated activity signal to the activity signal input unit 22. As one example, the activity sensor 30 may include an acceleration sensor. The acceleration sensor generates an activity signal by measuring a dynamic force such as acceleration, vibration, or impact. The acceleration sensor may employ various types of methods such as a method of measuring a dynamic force through an electromotive force generated in response to a conductor moving through a magnetic field, a method of measuring a dynamic force through a capacitance change with current, and a method of measuring a dynamic power through the Piezo-resistive effect of a semiconductor strain gauge. Furthermore, the acceleration sensor may also include a Micro Electro-Mechanical System (MEMS). Additionally, the activity sensor 30 may include a tilt sensor, a gyro sensor, the acceleration sensor, or any combination thereof.

The peak detecting unit 23 detects at least one of a plurality of peaks in a biological signal on the basis of an activity signal. The peak may indicate the highest point in the biological signal. As another example, the peak may be defined as maximum instantaneous points at which a biological signal changes from an increase to a decrease. Moreover, this peak may occur periodically in a biological signal having a predetermined period. For example, a P peak, an R peak, and a T peak may occur periodically in an ECG signal.

The peak detecting unit 23 detects one of a plurality of peaks in a biological signal in consideration of an activity signal. This detected peak may relate to an effective peak selected from a plurality of peaks. For example, in response to the biological signal being an ECG signal, the detected peak may mean an R peak selected from a plurality of peaks in the ECG signal. The peak detecting unit 23 will be described with reference to the accompanying drawings.

The output unit 24 outputs information regarding the detected peak. As an example, the information regarding the detected peak may include a peak occurrence time, a peak amplitude, and a peak time interval. Furthermore, the information regarding the detected peak may be delivered to the display device 40. Also, candidate detection peaks may be stored in the storage unit 25. The storage unit 25 will be described with reference to the accompanying drawings.

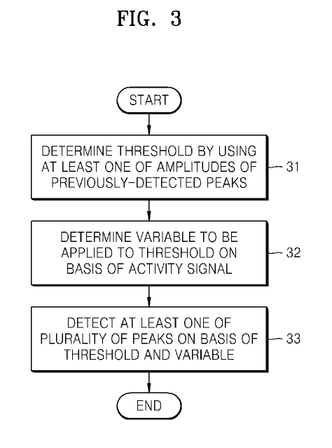

FIG. 3 is a flowchart illustrating an example of operations in response to the peak detecting unit 23 of FIG. 2 detecting a peak in consideration of an activity signal. Referring to FIG. 3, the detecting of the peak in consideration of the activity signal may include the following operations. In operation 31, the peak detecting unit 23 determines a threshold by using at least one of previously-detected peak amplitudes. The threshold may be an amplitude threshold determined from the previously-detected peak amplitudes. As another aspect, this threshold may be a threshold other than an amplitude threshold such as a slope threshold or a time interval threshold. According to another example, the peak detecting unit 23 may determine a threshold by using at least one of previously-detected peak slopes, and the threshold may refer to a slope threshold.

In operation 32, the peak detecting unit 23 determines a variable to be applied to a threshold on the basis of an activity signal. This variable may be used for changing the threshold. For example, in response to the threshold being determined to be 1 based on at least one of previously-detected peak amplitudes, the variable may be determined to be 0.4 and change the threshold from 1 to 0.4 by performing an operation on the threshold of 1. The operation may include multiplication, subtraction, etc. As another aspect, other examples may change a threshold by using a variable without performing an operation.

The variable may be determined on the basis of an activity signal. The peak detecting unit 23 may determine an amount of activity of the patient 50 based on an activity signal and determine a variable to change a threshold based on the determined amount of activity. For example, in response to the determined amount of activity being equal to or greater than a predetermined amount (for example, the patient 50 moves by more than a predetermined amount), the peak detecting unit 23 determines a variable to reduce a threshold.

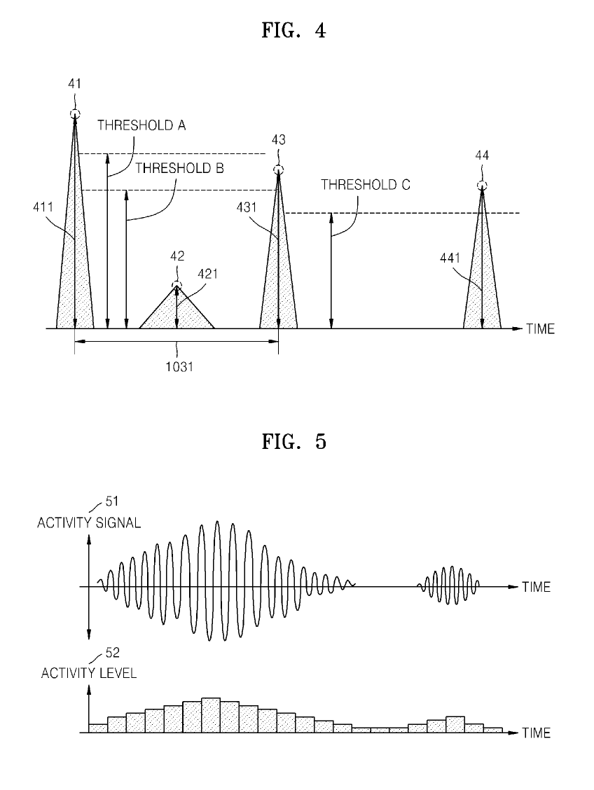

In operation 33, the peak detecting unit 23 detects at least one of a plurality of peaks in a biological signal on the basis of the determined threshold and variable. The peak detecting unit 23 may apply the determined variable on the determined threshold and detect at least one of a plurality of peaks on the basis of the variable and threshold. For example, in response to the threshold being determined as 1 and the variable being determined as 0.7, the peak detecting unit 23 detects at least one of a plurality of peaks on the basis of 0.7 (for example, the result obtained by multiplying 1 by 0.7). The peak detecting unit 23 may detect at least one of the plurality of peaks by comparing the determined variable being 0.7 with each of the plurality of peaks, which will be described with reference to FIG. 4.

FIG. 4 is a view illustrating an example of a biological signal. Operations 31 to 33 of FIG. 3 will be described with reference to FIG. 4. Referring to FIGS. 3 and 4, in operation 31, the peak detecting unit 23 determines a threshold A by using at least one of previously-detected peaks. The threshold A may be determined from the amplitude 422 of the most recent peak from among previously-detected peaks. For example, in response to the amplitude 411 of the detected peak 41 being 1.4, the threshold A may be determined as 2 (for example, the result obtained by dividing 1.4 by a proportional constant of 0.7). As another example, the threshold A may be determined by the amplitude of another detected peak or an average value of the amplitudes of a plurality of detected peaks.

Referring to FIGS. 3 and 4, in operation 32, the peak detecting unit 23 determines a variable to be applied to the threshold A on the basis of an activity signal. The variable may change the threshold A. For example, in response to an amount of activity determined from the activity signal being equal to or greater than a predetermined amount, the peak detecting unit 23 may determine the variable to be less than 1 in order to reduce the threshold A by multiplying the variable by the threshold A.

Referring to FIGS. 3 and 4, in operation 33, the peak detecting unit 23 detects a peak 43 from among the plurality of peaks 42 and 43 on the basis of the threshold and variable. For example, the peak detecting unit 23 may apply the variable to the threshold A to change the threshold A into a threshold B and detect the peak 43 by using the changed threshold, which is threshold B. As an example, in response to the threshold A being determined as 1 and the variable being determined to be 0.7, the peak detecting unit 23 determines the threshold B as 0.7 by multiplying the threshold A by the variable. After comparing the determined threshold being 0.7 with the amplitudes of the plurality of peaks 42 and 43, the peak detecting unit 23 detects the peak 43 having an amplitude 431 greater than the threshold B. At this point, since the peak 42 has an amplitude 421 less than the determined threshold B, the peak 42 may not be detected. The peak detecting unit 23 may prevent the case that the peak 43 may not be detected as the amplitude 411 of the previously-detected peak 41 is increased abnormally by a noise due to activity of the patient 50. In other words, the peak detecting unit 23 may take into consideration dynamic noise due to the activity of the patient 50 by dynamically changing a threshold for detecting a peak according to an amount of activity of the patient 50.

Referring to FIGS. 3 and 4, the peak detecting unit 23 may perform operations 31 to 33 at least once on peaks that occurred after the detected peak 43. In this case, the threshold may be determined on the basis of the amplitude 431 of the detected peak 43. For example, the peak detecting unit 23 determines a threshold C on the basis of the amplitude 431 of the peak 43 that occurred after the peak 42. In response to a determination that an amount of activity of the patient 50 is less than a predetermined threshold amount based on an activity signal, the peak detecting unit 23 does not determine a variable. As yet another aspect, in response to a determination that the amplitude 441 of an occurred peak 44 is equal to or greater than the threshold C, the peak detecting unit 23 detects the occurred peak 44. As another aspect, the variable may be determined as 1, and the variable may be applied to the threshold C.

According to various examples, the peak detecting unit 23 determines a threshold by using at least one of the slopes of the previously-detected peaks in operation 31. In operation 32, the peak detecting unit 23 determines a variable to be applied to the threshold on the basis of an activity signal. In operation 33, the peak detecting unit 23 may detect at least one of a plurality of peaks on the basis of the threshold and the variable. Regarding determining the slope of a peak, for example, the slope of a predetermined peak may mean a difference between a peak amplitude and the amplitude of a biological signal at a predetermined time interval. In other words, the slope of a predetermined peak may mean a degree of change in the peak. Furthermore, according to various examples, the peak detecting unit 23 determines a threshold by using at least one of the time intervals of the previously-detected peaks in operation 31. In operation 32, the peak detecting unit 23 determines a variable to be applied to the threshold on the basis of an activity signal. In operation 33, the peak detecting unit 23 may detect at least one of a plurality of peaks on the basis of the threshold and the variable. At this point, the threshold may be compared to each of the time intervals of a plurality of peaks. As an example, the time intervals between the plurality of peaks includes the time intervals between each of the previously-detected peaks and each of the plurality of peaks. Thus, the at least one of the plurality of peaks may be determined by using the threshold of a time interval.

As discussed, the peak detecting unit 23 determines an amount of activity of the patient 50 on the basis of an activity signal. The activity signal may have a value reflecting activity of the patient 50. For example, the activity signal may be numerically expressed as a value between 1 and 100 according to an amount of activity of the patient 50. The detecting unit 23 according to an example may determine a numerical value of the activity signal as the amount of activity of the patient 50. As another aspect, the peak detecting unit 23 according to another example may determine an activity level by using the activity signal and may determine the activity level as the amount of activity of the patient 50. Accordingly, other implementations for determining the amount of activity of the patient 50 from an activity signal are within the scope of the teachings herein.

FIG. 5 is a view illustrating examples of an activity signal and an amount of activity. Although there are various methods for determining an amount of activity by using an activity signal, one of the various methods will be described with reference to FIG. 5, for conciseness. As indicated by the reference number 51, the activity signal reflects the amount of activity of the patient 50, which changes over time. For example, the activity signal may be numerically expressed as values between 0 and 100 according to the amount of activity and thus may be changed between 0 and 100 over time so that the change in the amount of activity of the patient 50 may be reflected. The peak detecting unit 23 determines an activity level by accumulating the activity signal per unit of time. In other words, the peak detecting unit 23 may determine an activity level per unit of time by an integral of an activity signal per unit of time. For example, the peak detecting unit 23 determines an activity level based on an integral of an activity signal per 1 min unit of time. At this point, as indicated by the reference number 52, the activity level may reflect an activity change of the patient 50 as each 1 min unit of time passes.

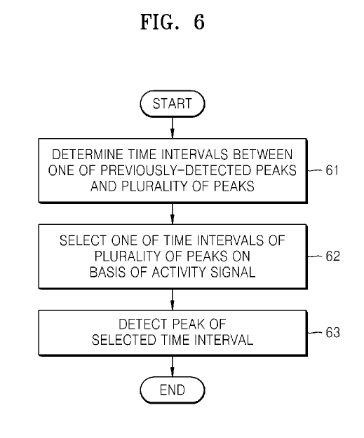

FIG. 6 is a flowchart illustrating an example of operations in response to the peak detecting unit 23 of FIG. 2 detecting a peak in consideration of an activity signal. Referring to FIG. 6, the detecting of the peak in consideration of the activity signal includes the following operations. In operation 61, the peak detecting unit 23 determines time intervals between one of previously-detected peaks and a plurality of peaks. The peak detecting unit 23 may determine time intervals between the peak detected at the closest time to the occurrence times of a plurality of peaks from among previously-detected peaks and the plurality of peaks. In other words, as a non-limiting example, the peak detecting unit 23 may determine a time interval of a plurality of peaks on the basis of the most recently occurred peak from among the detected peaks.

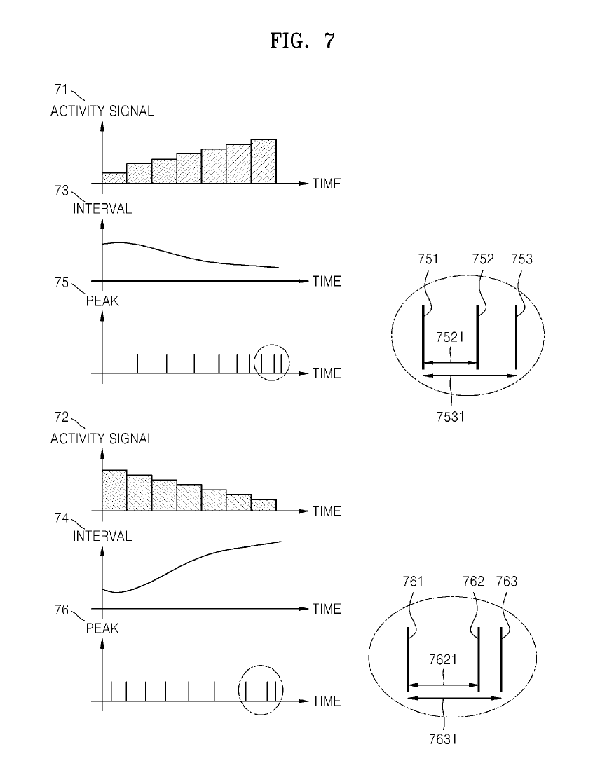

In operation 62, the peak detecting unit 23 selects one of the time intervals of the plurality of peaks on the basis of an activity signal. In operation 63, the peak detecting unit 23 detects a peak in the selected time interval among the plurality of peaks. The peak detecting unit 23 may determine a pattern that represents changes of a plurality of activity signals including the activity signal and determine one of the time intervals of the plurality of peaks on the basis of the determined pattern. For example, the peak detecting unit 23 selects one time interval that is greater than the time interval between previously-detected peaks from among the time intervals of a plurality of peaks in response to the pattern that represents an increased amount of activity of the patient 50 being determined on the basis of the plurality of activity signals including the activity signal. The peak detecting unit 23 determines an activity change characteristic of the patient 50, changes a criterion for selecting the time interval of the plurality of peaks on the basis of the activity change characteristic, and detects a peak on the basis of the changed criterion, so that peaks of a biological signal may be more accurately detected as the patient 50 moves around. At this point, the time intervals between the previously-detected peaks may include an average thereof, which will be described with reference to FIG. 7. FIG. 7 is a view illustrating examples of a pattern representing changes in the amount of activity of a patient, and a pattern representing time intervals and detected peaks. Referring to FIG. 7, the peak detecting unit 23 selects one time interval 7521 less than the time interval of the previously-detected peak 751 from among the time intervals of a plurality of peaks 752 and 753 in response to a pattern representing an increased amount of activity of the patient 50 on the basis of a plurality of activity signals including an activity signal. At this point, as indicated by reference number 71, the pattern representing the increased amount of activity of the patient 50 may be a pattern representing an increased activity level determined from the activity signals. Additionally, the time interval of the previously-detected peak 751 may mean the interval between the occurrence time of the detected peak 751 and the occurrence time of a peak detected right before the detected peak 751. The time interval of the peak 752 may correspond to the time interval 7521 between the occurrence time of the peak 752 and the occurrence time of the detected peak 751. The time interval of the peak 753 may correspond to the time interval 7531 between the occurrence time of the peak 753 and the occurrence time of the detected peak 751.

In response to the pattern representing an increased activity of the patient 50, the peak detecting unit 23 selects the time interval 7521 less than the time interval of the previously-detected peak 751 in anticipation of a pattern representing a gradually-decreased interval of the detected peaks from the patient 50. The peak detecting unit 23 may detect a peak more strongly than a dynamic noise due to activity of the patient 50. According to another example, in response to a determination that the pattern represents an increased amount of activity of the patient 50 on the basis of a plurality of activity signals including the activity signal, the peak detecting unit 23 selects the time interval 7521 of the peak 752 less than the time interval 7531 of the peak 753 from among the time intervals of the plurality of peaks 752 and 753, so that the pattern representing an increased activity of the patient 50 may be reflected in the peak detection. Additionally, the peak detecting unit 23 detects the peak 752 of the selected time interval 7521.

Referring to FIG. 7, in response to a determination that a pattern represents a decreased amount of activity of the patient 50 on the basis of a plurality of activity signals including the activity signal, the peak detecting unit 23 may select the time interval 7631 that is longer than the time interval of a previously-detected peak 761 from among the time intervals of a plurality of peaks 762 and 763. At this point, as indicated by the reference number 72, the pattern representing a decreased amount of activity of the patient 50 may be a pattern representing a decreased activity level determined from activity signals. Additionally, the time interval of the previously-detected peak 761 may mean an interval between the occurrence time of the detected peak 761 and the occurrence time of a peak detected right before the detected peak 761. The time interval of the peak 762 may mean the time interval 7621 between the occurrence time of the peak 762 and the occurrence time of the detected peak 761. The time interval of the peak 763 may mean the time interval 7631 between the occurrence time of the peak 763 and the occurrence time of the detected peak 761.

In response to the pattern representing a decreased activity of the patient 50, the peak detecting unit 23 selects the time interval 7631 that is greater than the time interval of the previously-detected peak 761 in anticipation of a pattern representing a gradually-increased interval of the detected peaks of the patient 50. The peak detecting unit 23 may detect a peak more strongly than a dynamic noise due to activity of the patient 50. According to another example, in response to a determination that the pattern represents a decreased amount of activity of the patient 50 on the basis of the plurality of activity signals including the activity signal, the peak detecting unit 23 selects the time interval 7631 of the peak 763 that is greater than a time interval 7621 of the peak 762 from among the time intervals of the plurality of peaks 762 and 763, so that a pattern representing a decreased activity of the patient 50 may be reflected on the peak detection. Additionally, the peak detecting unit 23 detects the peak 763 of the selected time interval 7631.

In operation 62, the peak detecting unit 23 according to the example determines a pattern representing a time interval change by using the time intervals between the previously-detected peaks and selects one of the time intervals of a plurality of peaks in consideration of a pattern representing a change in the amount of activity of the patient 50 and a pattern representing a time interval change. As indicated by the reference number 73 and the reference number 75, in response to a pattern representing an increased amount of activity of the patient 50, a pattern representing a decreased time interval of the previously-detected peaks may be shown. As indicated by the reference number 74 and the reference number 76, in response to a pattern representing decreased amount of activity of the patient 50, a pattern representing an increased time interval of the previously-detected peaks is shown. In other words, there is a correlation between the pattern representing a change in the amount of activity of the patient 50 and the pattern representing a time interval change of the previously-detected peaks. This correlation may be represented with a symmetrical characteristic. Accordingly, the peak detecting unit 23 may more effectively reduce possible errors in detecting peaks according to activity of the patient 50 in combination with the pattern representing a change in the amount of activity of the patient 50 and the pattern representing a time interval change of the previously-detected peaks. Furthermore, as shown in FIG. 7, a correlation between the pattern representing a time interval of the previously-detected peaks and the patterning representing a change in the amount of activity of the patient 50 may be flexibly used by the peak detecting unit 23. For example, activity of the patient 50 may be more accurately determined by using a pattern representing a decreased time interval of the previously-detected peaks, which is shown with a delay time after a pattern representing an increased activity level is shown.

Since operations 61 to 63 correspond to the above descriptions with reference to FIGS. 1 to 5, further descriptions are omitted for conciseness.

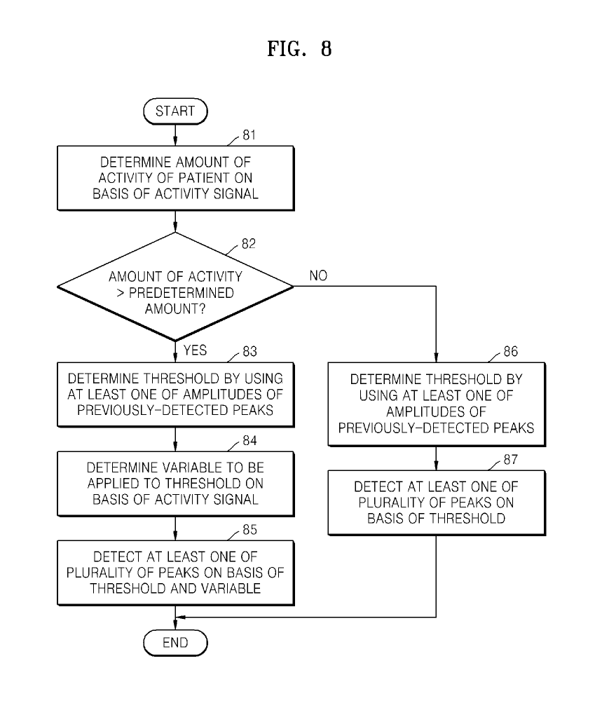

FIG. 8 is a flowchart illustrating another example of operations in response to the peak detecting unit 23 detecting a peak in consideration of an activity signal. Referring to FIG. 8, the detecting of the peak in consideration of the activity signal includes the following operations. In operation 81, the peak detecting unit 23 determines an amount of activity of the patient 50 on the basis of the activity signal. The activity signal may have a value corresponding to activity of the patient 50. For example, the activity signal may be numerically expressed with values between 0 and 100 according to an amount of activity of the patient 50. The peak detecting unit 23 may determine an amount of activity of the patient 50 on the basis of a numerical value of the activity signal. The peak detecting unit 23 of this example determines an activity level by using the activity signal and determines an amount of activity of the patient 50 by using the activity level. Accordingly, it is understood that other implementations are within the scope of the teachings herein for determining an amount of activity of the patient 50 from an activity signal. In operation 82, the peak detecting unit 23 compares the determined amount of activity with a predetermined amount. At this point, the amount of activity may be represented with the activity level. Also, the predetermined amount may mean a predetermined level.

In operation 83, in response to the amount of activity being greater than the predetermined amount, the peak detecting unit 23 determines a threshold by using at least one of the amplitudes of previously-detected peaks. In operation 84, the peak detecting unit 23 determines a variable to be applied to the threshold on the basis of the activity signal. In operation 85, the peak detecting unit 23 detects at least one of a plurality of peaks in the biological signal on the basis of the determined threshold and the determined variable. Since operations 83 to 85 correspond to those of operations 31 to 33, further descriptions are omitted for conciseness.

In operation 86, in response to the amount of activity being equal to or less than the predetermined amount, the peak detecting unit 23 determines a threshold by using at least one of the amplitudes of previously-detected peaks. Since operation 86 corresponds to that of operation 31 described with reference to FIGS. 3 to 5, further descriptions are omitted for conciseness. In operation 87, the peak detecting unit 23 determines at least one of a plurality of peaks on the basis of the determined threshold. In other words, in operation 87, in response to the amount of activity of the patient 50 being equal to or less than the predetermined amount, the peak detecting unit 23 detects at least one of a plurality of peaks by using the threshold determined from the previously-determined peak amplitude without consideration of a variable determined from the activity signal. The peak detecting unit 23 reflects the activity signal on the peak detection in response to the amount of activity being greater than the predetermined amount, and does not reflect the activity signal in the peak detection in response to the amount of activity not being greater than the predetermined amount, resulting in the stronger detection of a peak rather than a dynamic noise due to activity of the patient 50. Since descriptions related to operation 87 correspond to thatose described with reference to FIGS. 3 to 5, further descriptions are omitted for conciseness.

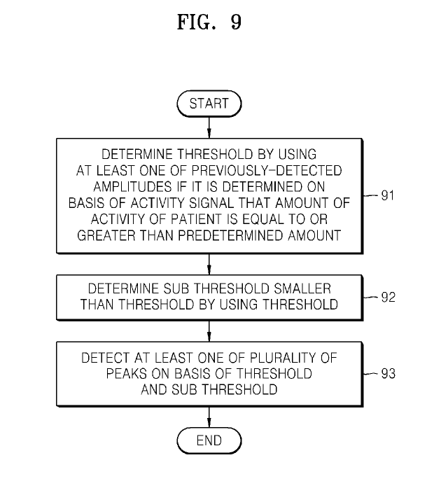

FIG. 9 is a flowchart illustrating another example of operations in response to the peak detecting unit 23 of FIG. 2 detecting a peak in consideration of an activity signal. Referring to FIG. 9, the detecting of the peak in consideration of the activity signal includes the following operations. In operations 91, in response to a determination on the basis of the activity signal that an amount of activity of the patient 50 is greater than a predetermined amount, the peak detecting unit 23 determines a threshold by using at least one of the amplitudes of previously-detected peaks. At this point, the amount of activity may mean an activity level and the predetermined amount may mean a predetermined level. In operation 91, the peak detecting unit 23 determines the threshold by using at least one of the amplitudes of the previously-detected peaks. The determining of the threshold is apparent on the basis of the description of operation 31.

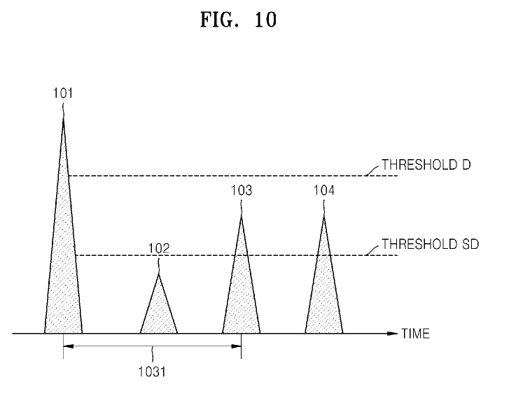

In operation 92, in response to a determination on the basis of the activity signal that the amount of activity of the patient 50 is greater than the predetermined amount, the peak detecting unit 23 determines a sub-threshold by using the threshold. In operation 93, the peak detecting unit 23 detects at least one of a plurality of peaks on the basis of the threshold and the sub-threshold. The sub-threshold may be less than the threshold. For example, in response to the threshold being a peak amplitude threshold and its value being 1, the sub-threshold may be determined as 0.7 less than the peak amplitude threshold. As another aspect, it is understood that this sub-threshold may include a slope sub-threshold determined from a slope threshold and a time interval sub-threshold determined from a time interval threshold. This will be described with reference to FIG. 10.

FIG. 10 is a view illustrating another example of a biological signal. Operations 92 and 93 are described with reference to FIG. 10. Although a threshold is described as a peak amplitude threshold hereinafter, it is understood that this is only a non-limiting example. For example, in other implementations, thresholds such as a slope threshold or a time interval threshold may be used. Referring to FIGS. 9 and 10, the amplitudes of peaks 102 to 104 that occur after a detected peak 101 are less than a threshold D (for example, an amplitude threshold determined from the amplitude of the detected peak 101). Accordingly, in response to the peak detecting unit 23 detecting a peak by using the threshold D, since the peaks 102 to 104 are less than the threshold D, no peak is detected from the peaks 102 to 104. Furthermore, although the peak 103 should be detected, the peak 103 is not detected due to activity of the patient 50. This detection error causes limitations in biological signal analysis.

In order to prevent the detection error, in operation 92, in response to the amount of activity of the patient 50 being equal to or greater than the predetermined amount, the peak detecting unit 23 further determines a threshold D (for example, an amplitude threshold determined from the amplitude of the detected peak 101) and a threshold SD (for example, a sub amplitude threshold determined from the threshold D). In operation 93, the peak detecting unit 23 detects one of the peaks 102 to 104 by using the threshold D and threshold SD. For example, in response to the amount of activity of the patient 50 being equal to or greater than the predetermined amount, the peak detecting unit 23 further determines the threshold SD. The threshold SD is determined based on a comparison of the amplitudes of the peaks 102 to 104 with the peak 101 due to activity of the patient 50 and detects the peak 103 having a larger amplitude than the threshold SD as a detection peak after comparing the determined threshold SD with the occurred peaks 102 to 104. At this point, the threshold SD may have a smaller value than the threshold D. For example, the threshold SD is ⅓ the amplitude of the threshold D. Additionally, the peak detecting unit 23 may detect the peak 103 from among the peaks 103 and 104 in consideration of a time interval 1031 of the peak 103. As another example, in response to the amount of activity of the patient 50 being equal to or greater than the predetermined amount, the peak detecting unit 23 further determines a sub-threshold besides a threshold for detection and uses the sub-threshold and the threshold to detect a peak, so that a method may be provided of detecting a peak more strongly than a noise due to the activity of the patient 50.

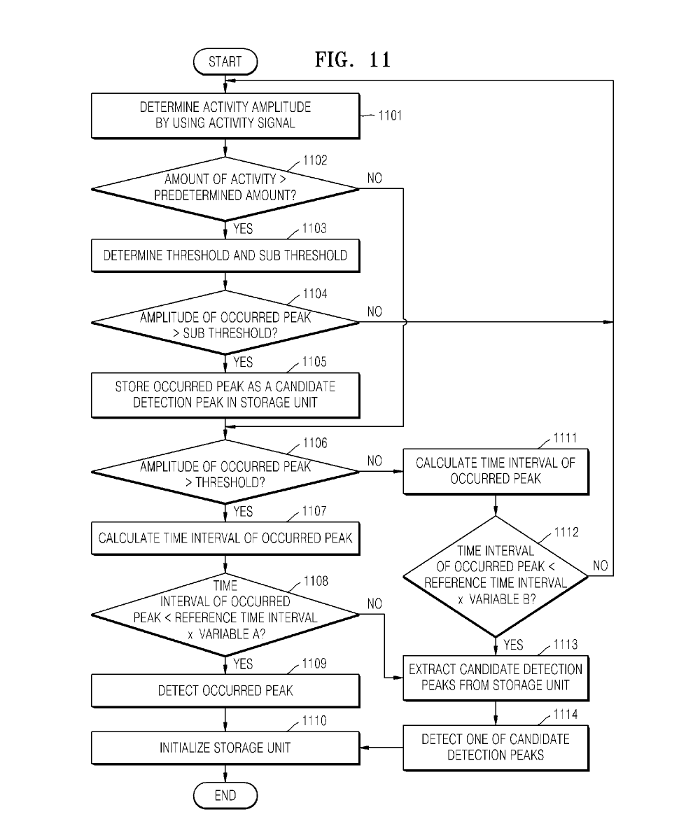

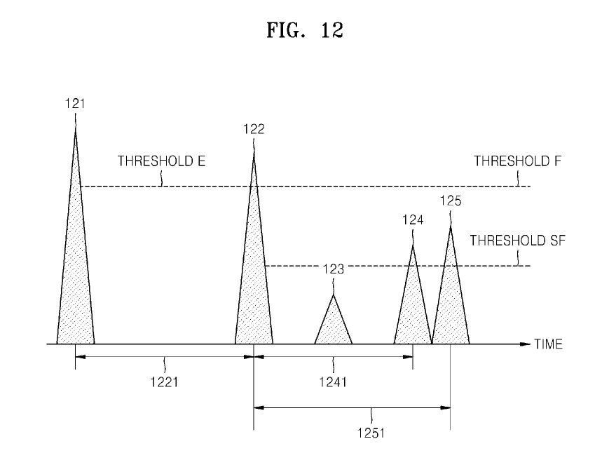

According to another example, in operation 93, in response to the amplitude of one of the plurality of peaks being equal to or greater than the sub-threshold, the peak detecting unit 23 stores the one peak as a candidate detection peak in the storage unit 25. In response to the amplitude of the one peak being equal to or greater than the threshold, the peak detecting unit 23 detects such a peak. In response to the amplitude of the one peak being equal to or greater than the sub-threshold and less than the threshold, the peak detecting unit 23 detects one of the candidate detection peaks stored in the storage unit 25. At this point, in response to the amplitude of the one candidate detection peak being equal to or greater than the sub-threshold and less than the threshold, the peak detecting unit 23 may detect one of a plurality of candidate detection peaks in consideration of a time interval between one peak and a previously-detected peak. The plurality of candidate detection peaks may include the candidate detection peak. Additionally, the one candidate detection peak may mean a currently-occurring peak from among a plurality of peaks. Operation 93 will be described with reference to FIGS. 11 and 12.

Since operations 91 to 93 correspond to those described with reference to FIGS. 1 to 8, further descriptions are omitted for conciseness.

FIG. 11 is a flowchart illustrating an example of operations in response to the peak detecting unit 23 detecting a peak in consideration of an activity signal. Referring to FIG. 11, the detecting of the peak in consideration of the activity signal includes the following operations. In operation 1101, the peak detecting unit 23 determines an activity level by using an activity signal. In operation 1102, the peak detecting unit 23 performs operation 1103 in response to the activity level being greater than a threshold level. As another aspect, in response to the activity level being equal to or less than the threshold level in operation 1102, the peak detecting unit 23 performs operation 1106. In operation 1103, the peak detecting unit 23 determines a threshold from the amplitude of the previously-detected peak and determines a sub-threshold from the threshold. For example, the threshold may include an amplitude or slope threshold and the sub-threshold may include a sub amplitude or slope threshold. The peak detecting unit 23 compares the sub-threshold and the amplitude of the occurred peak in operation 1104 and performs operation 1105 in response to the amplitude of the occurred peak being greater than the sub-threshold of the occurred peak. As another aspect, the peak detecting unit 23 compares the sub-threshold with the amplitude of the occurred peak and performs operation 1101 again in response to the amplitude of the occurred peak being equal to or less than the sub-threshold in operation 1104.

In operation 1105, the peak detecting unit 23 stores information regarding the occurred peak in the storage unit 25. At this point, the peak information stored in the storage unit 25 becomes one of at least one candidate detection peak stored in the storage unit 25. The peak detecting unit 23 compares the amplitude of the occurred peak with a threshold in operation 1106 and, in response to the amplitude being equal to or less than the threshold, performs operation 1111. In response to the amplitude not being equal to or less than the threshold, the peak detecting unit 23 calculates the time interval of the occurred peak in operation 1107. At this point, the time interval of the peak may mean the time interval between the occurred peak and the previously-detected peak. The peak detecting unit 23 compares the time interval of the occurred peak with the result obtained by multiplying a reference time interval by a variable A in operation 1108 and, in response to the time interval being less than the obtained result, the peak detection unit 23 detects the occurred peak as a detected peak in operation 1109. As another aspect, in response to the time interval being equal to or greater than the result in operation S1108, the peak detecting unit 23 extracts candidate detection peaks from the storage unit 25 in operation 1113. At this point, the variable A is one example applied to the time interval. Other examples may include a constant calculated from the reference time interval. In operation 1110, the peak detecting unit 23 initializes the storage unit 25 to delete the candidate detection peaks stored in the storage unit 25.

In operation 1111, the peak detecting unit 23 calculates the time interval of the occurred peak. The peak detecting unit 23 compares the time interval of the occurred peak with the result obtained by multiplying the time interval of the occurred peak by a variable B in operation 1112 and in response to the time interval of the occurred peak being less than the result, performs operation 1113. As another aspect, in response to the time interval of the occurred peak being equal to or greater than the result, the peak detecting unit 23 performs operation S1101 again. At this point, the variable B is one example applied to the time interval. As another example, variable B may be a constant calculated from the reference time interval. In operation S1113, the peak detecting unit 23 extracts at least one candidate detection peak stored in the storage unit 25. In operation 1114, the peak detecting unit 23 detects at least one peak among a plurality of peaks, as a detection peak, in consideration of an activity signal of the patient 50.

FIG. 12 is a view illustrating an example of a biological signal. The operations of FIG. 11 will be described with reference to FIG. 12. Referring to FIGS. 11 and 12, the peak detecting unit 23 determines an activity level by using an activity signal after the previously-detected peak 121. At this point, the peak detecting unit 23 may determine an activity level per unit of time in order to detect a peak after the previously-detected peak 121. The peak detecting unit 23 compares a threshold E (for example, an amplitude threshold determined from the amplitude of the previously-detected peak 121) with the amplitude of an occurred peak 122 and, in response to the amplitude of the occurred peak 122 being greater than the threshold E, the peak detecting unit 23 calculates the time interval 1221 of the occurred peak 122 in operation 1107. The time interval 1221 may mean the time interval between the previously-detected peak 121 and the occurred peak 122. The peak detecting unit 23 compares the time interval 1221 of the occurred peak 122 with the result obtained by multiplying the reference time interval by the variable A in operation 1108 and, in response to the time interval being less than the result, performs operation 1109. At this point, one example of the reference time interval includes an average value of the time intervals of the previously-detected peaks. As another example, the variable A may include a predetermined constant. For example, the variable A may be 1.5. In operation 1109, the peak detecting unit 23 detects the peak 122 as an effective peak. In operation 1110, the peak detecting unit 23 initializes the storage unit 25 to delete the candidate detection peaks stored in the storage unit 25.

Referring to FIGS. 11 and 12, the peak detecting unit 23 determines an activity level by using an activity signal after the previously-detected peak 122 in operation 1101. The peak detecting unit 23 compares the determined activity level with a threshold level in the operation 1102 and, in response to a determination that the activity level is greater than the threshold level, the peak detecting unit 23 determines a threshold F by using the previously-detected peak 122 and determines a threshold SF (for example, sub amplitude threshold) by using the threshold F in operation 1103. The peak detecting unit 23 compares the amplitude of an occurred peak 123 with the threshold SF in operation 1104 and, in response to the amplitude of the occurred peak 123 being equal to or less than the threshold SF, performs operation 1101 again.

Referring to FIGS. 11 and 12, the peak detecting unit 23 determines an activity level by using an activity signal after the previously-detected peak in operation 1101. The peak detecting unit 23 compares the determined activity level with a threshold level in operation 1102 and, in response to the activity level being greater than the threshold level, the peak detecting unit 23 determines a threshold F (for example, an amplitude threshold) by using the previously-detected peak 122 and determines a threshold SF (for example, a sub amplitude threshold) by using the threshold F in operation 1103. The peak detecting unit 23 compares the amplitude of the occurred peak 124 with the threshold SF in operation 1104 and, in response to the amplitude of the occurred peak 124 being greater than the threshold SF, the peak detecting unit 23 stores information regarding the occurred peak 124 in the storage unit 25 in operation 1105. At this point, the peak 124 stored in the storage unit 25 is one of at least one candidate detection peak stored in the storage unit 25. The peak detecting unit 23 compares the threshold F (for example, the amplitude threshold determined from the amplitude of the previously-detected peak 122) with the amplitude of the occurred peak 124 in operation 1106 and, in response to the amplitude of the occurred peak 124 being equal to or less than the threshold F, the peak detecting unit 23 calculates the time interval 1241 of the occurred peak 124 in operation 1111. This time interval 1241 may mean the time interval between the previously-detected peak 122 and the occurred peak 124. The peak detecting unit 23 compares the time interval 1241 of the occurred peak 124 with the result obtained by multiplying the time interval 1241 with the variable B in operation 1112 and, in response to the time interval 1241 of the occurred peak 124 being equal to or greater than the result, performs operation 1101. As another aspect, the occurred peak 124 as a candidate detection peak is stored in the storage unit 25.

Referring to FIGS. 11 and 12, the peak detecting unit 23 determines an activity level by using an activity signal after the previously-detected peak 122 in operation 1101. The peak detecting unit 23 compares the determined activity level with a threshold level in operation 1102 and, in response to the activity level being greater than the threshold level, the peak detecting unit 23 determines a threshold F (for example, an amplitude threshold) by using the previously-detected peak 122 and determines a threshold SF (for example, a sub amplitude threshold) by using the threshold F in operation 1103. The peak detecting unit 23 compares the amplitude of the occurred peak 125 with the threshold SF in operation 1104 and, in response to the amplitude of the occurred peak 125 being greater than the threshold SF, the peak detecting unit 23 stores information regarding the occurred peak 125 in the storage unit 25 in operation 1105. At this point, the peak 125 stored in the storage unit 25 is one of at least one candidate detection peak stored in the storage unit 25, and the previously-detected peak is also stored as a candidate detection peak in the storage unit 25. The peak detecting unit 23 compares the threshold F (for example, the amplitude threshold determined from the amplitude of the previously-detected peak 122) with the amplitude of the occurred peak 125 in operation 1106 and, in response to the amplitude of the occurred peak 125 being equal to or less than the threshold F, the peak detecting unit 23 calculates the time interval 1251 of the occurred peak 125 in operation 1111. The peak detecting unit 23 compares the time interval 1251 of the occurred peak 125 with the result obtained by multiplying the time interval 1241 by the variable B in operation 1112 and, in response to the time interval of the occurred peak 125 being less than the result, performs operation 1113. At this point, one example of the reference time interval includes an average value of the time intervals between the previously-detected peaks. Additionally, the variable B may be determined as 1.5. As another aspect, this variable B may be determined as being different from the variable A so that the upper limit and lower limit for peak detection can be provided. In operation 1113, the peak detecting unit 23 extracts at least one candidate peak from the storage unit 25. At this point, the candidate detection peak includes the peak 124 and the peak 125. In operation 1114, the peak detection unit 23 detects one of the candidate detection peaks as a detection peak. For example, in operation 1114, the peak detection unit 23 detects the peak 125 as a detection peak on the basis that the amplitude of the peak 125 is greater than that of the peak 124. As another example, the peak detecting unit 23 may determine one of the peaks 124 and 125 as one detection peak by using other factors such as the time interval and slopes of the peaks 124 and 125 in operation 1114. Additionally, the peak detecting unit 23 determines one of the peaks 124 and 125 as a detection peak on the basis of at least one of the pattern representing an amount of activity change of the patient 50 and the pattern representing a time interval change of the previously-detected peaks. In operation 1110, the peak detecting unit 23 initializes the storage unit 25 to delete the candidate detection peaks stored in the storage unit 25 in operation 1105. The peak detecting unit 23 detects one of a plurality of occurred peaks in consideration of the amount of activity of the patient 50, so that peak detection may be achieved with minimal errors even in response to the patient 50 moving around. As another example, in response to the time interval of the occurred peak being less than the obtained result in operation 1112, the peak detecting unit 23 detects one of the candidate detection peaks as a detection peak in consideration of the time interval between the occurred peak and the previously-detected peak in response to no peak being detected within the reference time interval. An error whereby the peak to be actually detected is not detected due to activity of the patient 50 can be prevented.

The peak detecting unit 23 according to an example determines a pattern representing a change in the amount of activity of the patient 50 on the basis of the activity signal and detects one of a plurality of candidate detection peaks on the basis of the determined pattern in operation 1114. Additionally, the peak detecting unit 23 according to another example determines a pattern representing a time interval change by using the time intervals between the previously-detected peaks and detects one of a plurality of candidate detection peaks on the basis of the pattern representing a change in the amount of activity of the patient 50 and the pattern representing a time interval change. Operation 1114 is understood from the description regarding the detecting of the one of the plurality of peaks on the basis of the pattern representing a change in the amount of activity and the pattern representing a time interval change. Thus, a further description of operation 1114 is omitted for conciseness.

Each time a peak occurs and in response to the occurred peak being greater than a sub-threshold, the peak detecting unit 23 stores the occurred peak in the storage unit 25 in real time in operation 1105, so that a resource used to perform a search back operation for detecting a peak can be saved for some other use. During the peak detection, the search back operation is a process for detecting a peak by re-searching peaks that occurred after a previously-detected peak in response to the peak not being detected during a predetermined time after the previously-detected peak. Since the search back operation requires all signal data including occurred peaks to be stored for re-searching the occurred peaks after a previously-detected peak, a resource may be wasted. As another aspect, the peak detecting unit 23 determines peaks having an amplitude equal to or greater than a sub-threshold from among the occurred peaks, as candidate detection peaks and stores information regarding the candidate detection peaks in the storage unit 50 in real time, so that a resource may be effectively used. Furthermore, the storage unit 50 may be initialized in response to one of the candidate detection peaks being detected and thus, may operate more effectively. Additionally, the information regarding candidate detection peaks may include occurrence times of peaks, amplitudes of peaks, and time intervals of peaks.

Since operations 1101 to 1114 correspond to those described with reference to FIGS. 1 to 10, further descriptions are omitted for conciseness.

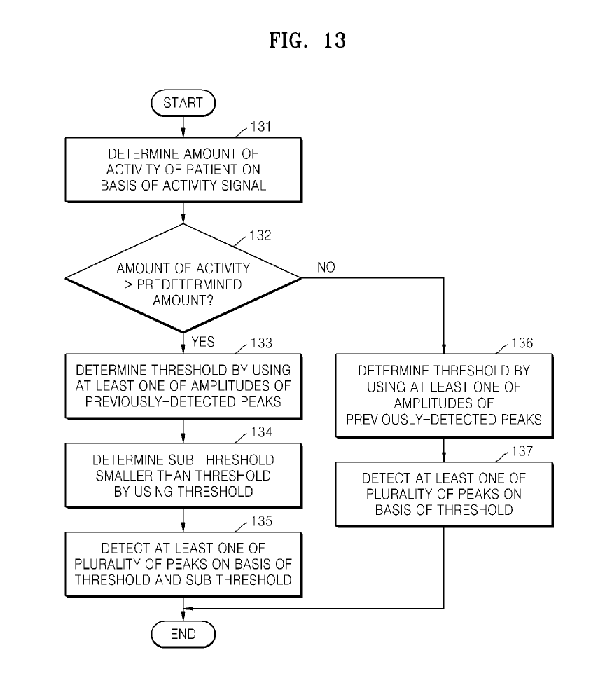

FIG. 13 is a flowchart illustrating another example of operations in response to the peak detecting unit 23 detecting a peak in consideration of an activity signal. Referring to FIG. 13, the detecting of the peak in consideration of the activity signal includes the following operations. In operation 131, the peak detecting unit 23 determines an amount of activity of the patient 50 on the basis of the activity signal. The activity signal may have a value reflecting activity of the patient 50. For example, the activity signal may be numerically expressed with values between 0 and 100 according to an amount of activity of the patient 50. The peak detecting unit 23 may determine an amount of activity of the patient 50 on the basis of a numerical value of the activity signal. The peak detecting unit 23 according to an example determines an activity level by using the activity signal and determines an amount of activity of the patient 50 by using the activity level. Accordingly, other implementation are within the scope of the teachings herein for determining an amount of activity of the patient 50 from an activity signal. In operation 132, the peak detecting unit 23 compares the determined amount of activity with a predetermined amount. At this point, the amount of activity may be represented with the activity level. In the same context, the predetermined amount may correspond with a predetermined level.

In operation 133, in response to the amount of activity being greater than the predetermined amount, the peak detecting unit 23 determines a threshold by using at least one of the amplitudes of previously-detected peaks. In operation 134, the peak detecting unit 23 determines a sub-threshold less than the threshold by using the threshold. In operation 135, the peak detecting unit 23 detects at least one of a plurality of peaks by using the threshold and the sub-threshold. Since operations 133 to 135 correspond to those of operations 91 to 93 described with reference to FIGS. 9 to 12, further descriptions are omitted for conciseness.

In operation 136, in response to the amount of activity being equal to or less than the predetermined amount, the peak detecting unit 23 determines a threshold by using at least one of the amplitudes of previously-detected peaks. Since operation 136 corresponds to that of operation 31 described with reference to FIGS. 3 to 5, further descriptions are omitted for conciseness. In operation 137, the peak detecting unit 23 detects at least one of a plurality of peaks on the basis of the determined threshold. In other words, in response to the amount of activity of the patient 50 being equal to or less than the predetermined amount, the peak detecting unit 23 detects at least one of a plurality of peaks by using the threshold determined from the previously-determined peak amplitude without consideration of the sub-threshold besides the threshold in operation 137. In response to the amount of activity being greater than the predetermined amount, the peak detecting unit 23 reflects the threshold and the sub-threshold in the peak detection and, in response to the amount of activity not being greater than the predetermined amount, the peak detecting unit 23 does not reflect them, resulting in stronger detection of a peak rather than a dynamic noise due to activity of the patient 50. Since operation 137 corresponds to that described with reference to of FIGS. 1 to 12, further descriptions are omitted for conciseness.

According to another example, in response to it being determined on the basis of the activity signal that the amount of activity of the patient 50 is greater than the predetermined amount of activity, the peak detecting unit 23 may detect one of at least one candidate detection peak stored in the storage unit 25, as a detection peak. For example, the peak detecting unit 23 may detect a peak having the largest amplitude from among the candidate detection peaks, as a detection peak. As another aspect, the peak detecting unit 23 may determine one of the candidate detection peaks as a detection peak by using factors such as a time interval and a slope of each of the candidate detection peaks. Additionally, the peak detecting unit 23 may determine one of the candidate detection peaks as a detection peak on the basis of at least one of the pattern of the activity levels and the pattern of the time intervals of the previously-detected peaks. This candidate detection peak may be one of the peaks, which are occurred and stored in the storage unit 25 in real time, as mentioned above. The peak detecting unit 23 detects one of the candidate detection peaks as a detection peak.

According to another example, in response to the amount of activity of the patient 50 being greater than a predetermined amount on the basis of the activity signal, the peak detecting unit 23 selects one of detection algorithms and detects at least one of a plurality of peaks on the basis of the selected detection algorithm. At this point, examples of the selected detection algorithm include an algorithm using operations 31 to 33 of FIG. 3, an algorithm using operations 61 to 63 of FIG. 6, an algorithm using operations 91 to 93 of FIG. 9, and an algorithm using operations 1101 to 1114 of FIG. 11. Additionally, in response to the amount of activity of the patient 50 being equal to or less than the predetermined amount on the basis of the activity signal, the peak detecting unit 23 selects another one of the detection algorithms and detects at least one of the plurality of peaks on the basis of the selected other one of the detection algorithms. At this point, examples of the other one of the detection algorithms include an algorithm using operations 86 and 86 of FIG. 8 and an algorithm using operations 136 and 137 of FIG. 13.

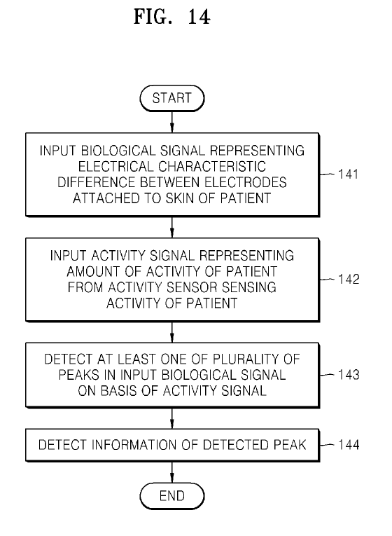

FIG. 14 is a flowchart illustrating an example of a peak detecting method. The peak detection method of FIG. 14 includes time-series operations processed in the peak detecting device 20 shown in FIG. 1.

In operation 141, the biological signal input unit 21 receives from the biological signal detecting device 10 a biological signal representing an electrical characteristic difference between electrodes attached to the skin of the patient 50. In operation 142, the activity signal input unit 22 receives an activity signal representing an amount of activity of the patient 50 from the activity sensor 30 sensing activity of the patient 50. In operation 143, the peak detecting unit 23 detects at least one of a plurality of peaks in the biological signal input on the basis of the activity signal. In operation 144, the output unit 24 outputs information regarding the detected peak.

Since a peak of the biological signal is determined as a detection peak in consideration of the activity signal, a peak may be detected from the biological signal in a noisy environment caused from activity of the patient 50. Additionally, since a peak is detected in the noisy environment, a portable biological signal measuring device for monitoring and analyzing a vital signal of the patient 50 in real time may be provided.

In addition, the peak detecting method of FIG. 14 may also be implemented through computer readable code/instructions in/on a medium, e.g., a computer readable medium, to control at least one processing element to implement any of the above described embodiments. The medium may correspond to any medium/media permitting the storage and/or transmission of the computer readable code.

An example of a medical device including the biological signal measuring device includes an electrocardiograph and an electromyograph.

Program instructions to perform a method described herein, or one or more operations thereof, may be recorded, stored, or fixed in one or more computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein. Also, the described unit to perform an operation or a method may be hardware, software, or some combination of hardware and software. For example, the unit may be a software package running on a computer or the computer on which that software is running. A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A peak detecting method comprising:
receiving a biological signal that represents an electrical characteristic difference between electrodes attached to a patient;
receiving an activity signal that represents an amount of activity of the patient from an activity sensor detecting activity of the patient;
detecting at least one of a plurality of peaks in the input biological signal on the basis of the activity signal; and
outputting information regarding the detected peak.

2. The peak detecting method of claim 1, wherein the detecting of the at least one of the plurality of peaks comprises:
determining a time interval between one of previously-detected peaks and each of the plurality of peaks;
selecting one of time intervals of the plurality of peaks on the basis of the activity signal; and
detecting a peak of the selected time interval from among the plurality of peaks.

3. The peak detecting method of claim 2, wherein the detecting of the at least one of the plurality of peaks comprises:
determining a pattern that represents a change in an amount of activity of the patient on the basis of a plurality of activity signals including the activity signal; and
selecting one of the time intervals of the plurality of peaks on the basis of the determined pattern.

4. The peak detecting method of claim 3, wherein the detecting of the at least one of the plurality of peaks comprises:
in response to it being determined on the basis of the plurality of activity signals including the activity signal that the pattern represents an increased amount of activity of the patient, determining a previous time interval by using time intervals between previously-detected peaks; and
selecting one time interval that is less than the previous time interval from among the time intervals of the plurality of peaks.

5. The peak detecting method of claim 3, wherein the detecting of the at least one of the plurality of peaks comprises:
in response to it being determined on the basis of the plurality of activity signals including the activity signal that the pattern represents a decreased amount of activity of the patient, determining a previous time interval by using the time intervals between previously-detected peaks; and
selecting one time interval that is greater than the previous time interval from among the time intervals of the plurality of peaks.

6. The peak detecting method of claim 4, wherein the previous time interval is an average of the time intervals between the previously-detected peaks.

7. The peak detecting method of claim 3, wherein the detecting of the at least one of the plurality of peaks comprises:
determining a pattern that represent a time interval change by using time intervals of previously-detected peaks;
selecting one of the time intervals of the plurality of peaks on the basis of the pattern that represents the change in the amount of activity of the patient and the pattern that represents the time interval change; and
detecting a peak of the time interval selected from the plurality of peaks.

8. The peak detecting method of claim 1, wherein the detecting of the at least one of the plurality of peaks comprises:
determining a threshold by using at least one of amplitudes of previously-detected peaks;
determining a variable to be applied to the threshold on the basis of the activity signal; and
detecting at least one of the plurality of peaks on the basis of the threshold and the variable.

9. The peak detecting method of claim 8, wherein the detecting of the at least one of the plurality of peaks comprises:
in response to it is determined on the basis of the activity signal that an amount of activity of the patient is equal to or greater than a predetermined amount, determining the variable to reduce the threshold.

10. The peak detecting method of claim 1, wherein the detecting of the at least one of the plurality of peaks comprises:
determining an amount of activity of the patient on the basis of the activity signal;
in response to the amount of activity of the patient being equal to or greater than a predetermined amount, determining a threshold by using at least one of amplitudes of previously-detected peaks, determining a variable to be applied to the threshold on the basis of the activity signal, and detecting at least one of the plurality of peaks on the basis of the threshold and the variable; and
in response to the amount of activity of the patient being less than the predetermined amount, determining a threshold by using at least one of amplitudes of previously-detected peaks and detecting at least one of the plurality of peaks on the basis of the threshold.

11. The peak detecting method of claim 1, wherein the detecting of the at least one of the plurality of peaks comprises:
in response to it being determined on the basis of the activity signal that the amount of activity of the patient is equal to or greater than a predetermined amount, determining a threshold by using at least one of amplitudes of previously-detected peaks, determining a sub-threshold less than the threshold by using the threshold, and detecting at least one of the plurality of peaks on the basis of the threshold and the sub-threshold.

12. The peak detecting method of claim 11, wherein the detecting of the at least one of the plurality of peaks comprises:
in response to it being determined on the basis of the activity signal that the amount of activity of the patient is equal to or greater than a predetermined amount, determining a threshold by using at least one of amplitudes of previously-detected peaks, and determining a sub-threshold less than the threshold by using the threshold;
in response to one of the plurality of peaks having an amplitude equal to or greater than the sub-threshold, storing the one peak as a candidate detection peak;
in response to the one peak having an amplitude equal to or greater than the threshold, detecting the one peak; and
in response to the one peak having an amplitude less than the threshold, detecting one of a plurality of candidate detection peaks including the candidate detection peak.

13. The peak detecting method of claim 12, wherein the storing of the one peak as the candidate detection peak comprises:
in response to one peak having an amplitude equal to or greater than the sub-threshold occurs, storing the one peak as the candidate detection peak in real time.

14. The peak detecting method of claim 12, wherein the detecting of the one of the plurality of candidate detection peaks comprises:
determining a pattern that represents a change in the amount of activity of the patient on the basis of a plurality of activity signals including the activity signal; and
determining one of the plurality of candidate detection peaks on the basis of the determined pattern.

15. The peak detecting method of claim 14, wherein the detecting of the one of the plurality of candidate detection peaks comprises:
determining a pattern that represents a time interval change by using time intervals between previously-detected peaks; and
detecting one of the plurality of candidate detection peaks on the basis of the pattern that represents the change in the amount of activity of the patient and the pattern that represents the time interval change.

16. The peak detecting method of claim 12, wherein the detecting of the one of the plurality of candidate detection peaks comprises:
in response to the one peak being less than the threshold, detecting one of a plurality of candidate detection peaks including the candidate detection peak in consideration of a time interval between the one peak and a previously-detected peak.

17. The peak detecting method of claim 11, wherein the detecting of the one of the plurality of peaks comprises:
determining an amount of activity of the patient on the basis of the activity signal;
in response to the amount of activity of the patient being equal to or greater than a predetermined amount, determining a threshold by using at least one of amplitudes of previously-detected peaks, determining a sub-threshold less than the threshold by using the threshold, and detecting at least one of the plurality of peaks on the basis of the threshold and the sub-threshold; and
in response to the amount of activity of the patient being less than the predetermined amount, determining a threshold by using at least one of amplitudes of previously-detected peaks and detecting at least one of the plurality of peaks on the basis of the threshold.

18. The peak detecting method of claim 11, wherein the detecting of the one of the plurality of peaks comprises selecting at least one effective peak from among a plurality of peaks in the input biological signal on the basis of the activity signal.

19. A non-transitory computer readable recording medium on which a program for executing the method of claim 1 is stored.

20. A peak detecting device comprising:
a biological signal input unit configured to receive a biological signal that represents an electrical characteristic difference between electrodes attached to a patient;
an activity signal input unit configured to receive an activity signal that represents an amount of activity of the patient from an activity sensor detecting activity of the patient;
a peak detecting unit configured to detect at least one of a plurality of peaks in the input biological signal on the basis of the activity signal; and
an output unit configured to output information regarding the detected peak.

21. The peak detecting method of claim 1, wherein the activity signal is separate from the input biological signal.

22. The peak detecting method of claim 1, wherein the activity signal represents acceleration, vibration, or impact.

23. A medical device comprising:
a peak detecting unit comprising:
an activity signal input unit configured to receive an activity signal that represents an amount of activity of a patient; and
a peak detector configured to detect at least one of a plurality of peaks in an input biological signal on the basis of the activity signal.

24. The medical device of claim 23 further comprising a display configured to display the information regarding the at least one detected peak.
